(19) United States
(12) Patent Application Publication
(10) Pub. No.: US 2012/0245595 A1
(43) Pub. Date: Sep. 27, 2012

(54) System and Method for Endovascular Telerobotic Access

(75) Inventors: Thenkurussi Kesavadas, Clarence Center, NY (US); Govindarajan Srimathveeravalli, Astoria, NY (US)

(73) Assignee: The Research Foundation of State University of New York, Amherst, NY (US)

(21) Appl. No.: 13/391,764

(22) PCT Filed: Aug. 26, 2010
(86) PCT No.: PCT/US10/46873

§ 371 Date: Jun. 12, 2012

Related U.S. Application Data
(60) Provisional application No. 61/237,163, filed on Aug. 26, 2009.

(51) Int. Cl. A61B 19/00

(52) U.S. Cl. 606/130

(57) ABSTRACT

A system for manipulating elongate surgical instruments comprises a console, which comprises an input controller. The input controller may have a haptic feedback mechanism. The system further comprises a slave component, which comprises a first linear actuator, a second linear actuator, and a first rotational actuator. Each actuator is in electrical communication with the input controller. The slave component further comprises a force sensor in electronic communication with the input controller. The force sensor is configured to measure a force acting upon the first elongate member on at least one degree of freedom. The force sensor will send a force signal to the haptic feedback mechanism of the input controller.

### CROSS-REFERENCE TO RELATED APPLICATION

This application claims the benefit of priority to U.S. provisional patent application Ser. No. 61/237,163 filed Aug. 26, 2009, now pending, the disclosure of which is incorporated herein by reference.

### FIELD OF THE INVENTION

The invention relates to generally to remote techniques for minimally-invasive surgery, and more particularly to a system and method for endovascular telerobotic access.

### BACKGROUND OF THE INVENTION

Surgeons face a number of unique challenges when carrying out endovascular procedures. Because they are no longer in direct contact with the site of operation, endovascular procedures represent a major paradigm shift away from open surgery. Treatments such as angioplasty using stents are almost never carried out through open surgery any longer. The surgical tools being flexible and elongated have dynamics of motion that is difficult to predict. There is no direct visual feedback of the operated site and all visual information is made available through a sequence of 2-D X-Rays or reconstructed 3D geometries. The surgeon only experiences the proximal forces on the tool and does not experience forces at the point of interaction between tool tip and vasculature. Similar to laparoscopy, hand movements and corresponding tool movement can be in different directions, for example, pulling on a guidewire may in fact cause it to elongate and advance into a vessel. Also, due to the high flexibility of the interventional device and the tortuous nature of vasculature, the tool behavior cannot be accurately predicted at any point in time. There can be significant variations in torque transmission in guidewires, making precise steering difficult. Thus, the motor skill set required for endovascular surgery is very different from that of open surgery and takes many years of specific training to master. Some other challenges that the surgeon faces when performing endovascular surgery include miniscule hand movements needed to steer tools, precision control of tools, hand tremor and any lack of dexterity is amplified manifold. In all it is very difficult to master and perform successfully.

Endovascular surgery and a few other forms of MIS techniques are carried out in a fluoroscopic suite. Because of this the surgeons receive continuous and daily exposure to radiation and have to wear heavy lead aprons during the procedure. This creates a continuous occupational hazard for the surgeon and can cause considerable discomfort when carrying out the surgery.

### BRIEF SUMMARY OF THE INVENTION

A system for manipulating elongate surgical instruments comprises a console, which comprises an input controller. The input controller may have a haptic feedback mechanism. The system further comprises a slave component, which comprises a first linear actuator, a second linear actuator, and a first rotational actuator. Each actuator is in electrical communication with the input controller. The slave component further comprises a force sensor in electronic communication with the input controller. The force sensor is configured to measure a force acting upon the first elongate member on at least one degree of freedom (“d.o.f.”). The force sensor will send a force signal to the haptic feedback mechanism of the input controller.

A system of the present invention can be used for any application that requires guiding and positioning long tubular structures inside bodily lumen, including, but not limited to:
Endoscopy, including colonoscopy, and bronchoscopy
Neurvascular surgery
Cardiology, cardiac interventions
Urology

### DESCRIPTION OF THE DRAWINGS

For a fuller understanding of the nature and objects of the invention, reference should be made to the following detailed description taken in conjunction with the accompanying drawings, in which:

FIG. 1A depicts a system according to an embodiment of the present invention;

FIG. 1B depicts the system of FIG. 1A being operated by a user;

FIG. 5 is a schematic of the components and features of one embodiment of the present invention;

FIG. 6 depicts the degrees of freedom of an interventional tool;

FIG. 7 depicts points at which interventional tools are handled by surgeons during a procedure and the corresponding type of actuation;

FIG. 8 depicts the kinematics of a section of an interventional tool, held rigid;

FIG. 2A depicts a first linear actuator and second linear actuator of a slave component according to an embodiment of the present invention;

FIG. 2B depicts a mounting arm of a slave component according to an embodiment of the present invention;

FIG. 2C depicts a first rotational actuator of a slave component according to an embodiment of the present invention;

FIG. 9 is a schematic of components of a friction wheel drive;

FIG. 10 depicts free body force diagrams for the drive wheel and catheter of a first linear actuator;

FIG. 3 depicts a first rotational actuator according an embodiment of the present invention using a friction wheel drive;

FIG. 4 depicts a first rotational actuator according to another embodiment of the present invention using a miniature gripper;

FIG. 11 is a schematic of the steering mechanism (gripper) and a free body diagram of a catheter inside the gripper;

FIG. 12 depicts a linear actuator according to one embodiment of an exemplary system;

FIG. 13 depicts a miniature gripper used for transmitting torsion to interventional tools;

FIG. 14A depicts a pulley used to transfer a driving force to a first rotational actuator;

FIG. 14B is another view of the pulley of FIG. 14A;

FIG. 15A is a view of a traveling cart;

FIG. 15B is another view of the traveling cart of FIG. 15A;

FIG. 16 shows a slave component of an exemplary system;

FIG. 17 is a cabling and wiring diagram for a servomotor (image courtesy EPOS getting started guide);

FIG. 18 is a graph of results from a PID Controller tuning for servomotor using Maxon's EPOS user software (top: linear actuator and bottom: rotational actuator), wherein the x axes represent time and the y axes represent velocity (encoder/ms);

FIG. 19 is a wiring diagram for a force sensor;

FIG. 20 is a graph showing force sensor calibration curves;

FIG. 21 shows control and sensing electronics used for a slave manipulator (top photo); an EPOS servo controller (bottom right photo); and a DGH data acquisition system (bottom left photo);

FIG. 22 shows a drive train for servo mechanisms;

FIG. 23 shows positioning and velocity following accuracy of a linear drive;

FIG. 24 shows positioning accuracy of a steering mechanism;

FIG. 25 shows a Novint Falcon haptic device (image courtesy of Warped Sounds blog);

FIG. 26 shows a mapping of Falcon to slave manipulators;

FIG. 27 shows a schematic of teleoperation in SETA;

FIG. 28 shows a schematic of a unilateral teleoperation control;

FIG. 29A shows results from PD controller used for teleoperation (linear actuator);

FIG. 29B shows results from PD controller used for teleoperation (rotational actuator);

FIG. 30 is a schematic of an impedance controller used for haptic feedback;

FIG. 31A shows haptic forces experienced by a used when inserting a guidewire into a vascular phantom;

FIG. 31B shows haptic forces experienced by a used when inserting a guidewire into a vascular phantom;

FIG. 32 depicts a menu for altering motion scaling;

FIG. 33 shows a comparison of smoothed and raw haptic feedback forces;

FIG. 34 depicts a software architecture for an exemplary system;

FIG. 35 depicts a system according to another embodiment of the present invention; and

FIG. 36 depicts a method according to an embodiment of the present invention.

### DETAILED DESCRIPTION OF THE INVENTION

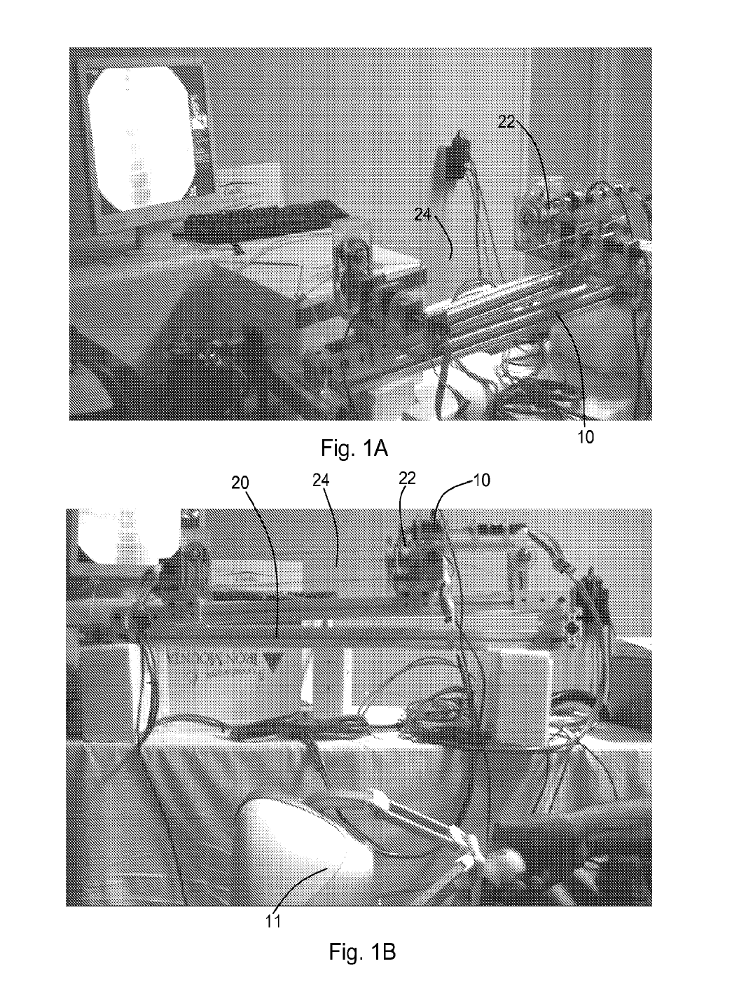

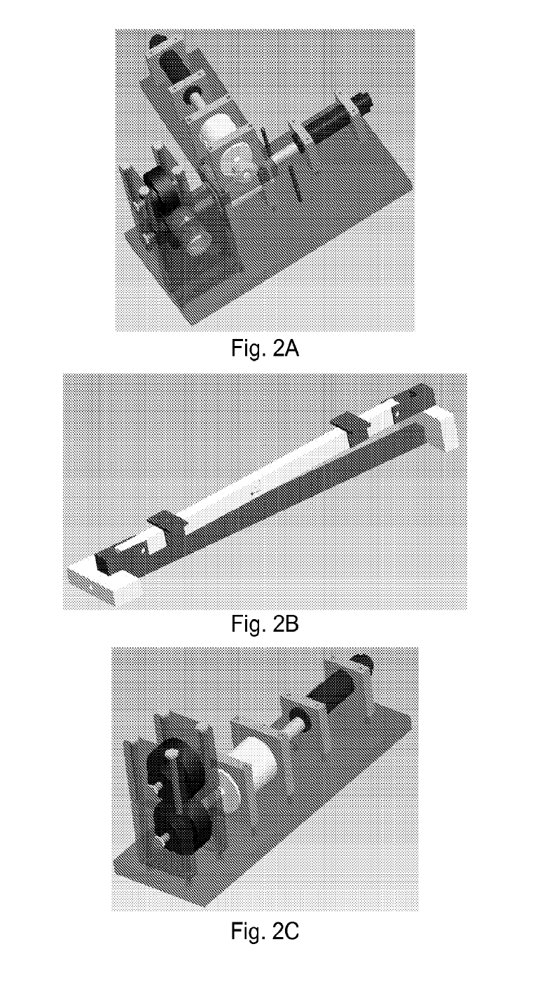

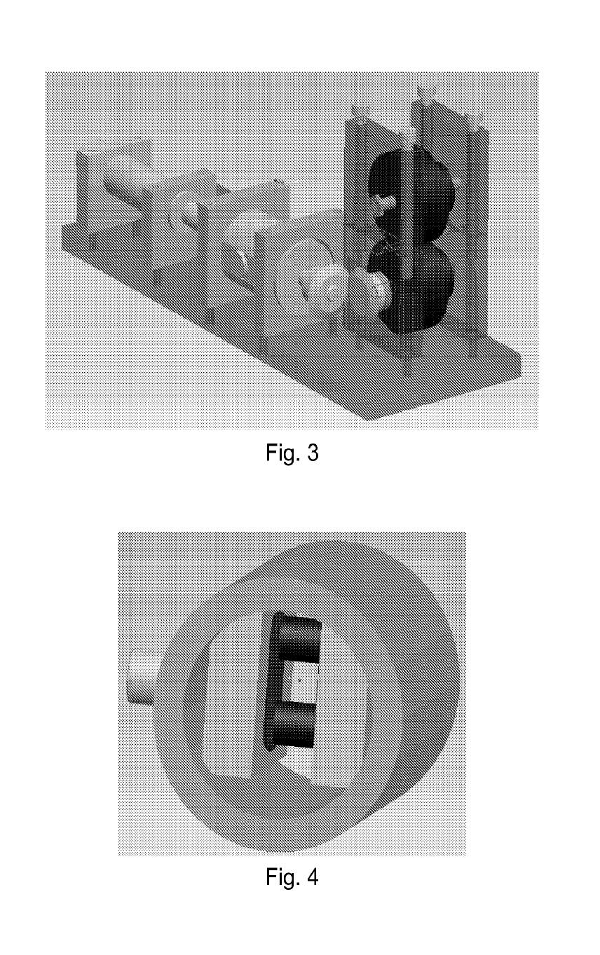

Reference is now made to FIGS. 1A and 1B, wherein a system 10 for manipulating elongate surgical instruments such as, but not limited to endovascular instruments according to an embodiment of the present invention is depicted. The system 10 is capable of manipulating at least two elongate instruments. The instruments may be, for example but not limited to, a guidewire and a catheter. The instruments may be coaxial, such that, for example, the guidewire may pass through a cavity of the catheter.

The system 10 comprises a console 11, which comprises an input controller 12. The input controller 12 is operable by a user, for example, a surgeon. The input controller 12 may have a haptic feedback mechanism such that a user will be able to sense forces produced by the haptic feedback mechanism.

The system 10 further comprises a slave component 20, which comprises a first linear actuator 22 (see, e.g., FIG. 2A). The first linear actuator 22 is in electronic communication with the input controller 12 such that the first linear actuator 22 receives a first translatory signal (not shown) sent from the input controller 12. The first linear actuator 22 is configured to cause a motion (translation) of a first elongate instrument 24. The first linear actuator 22 will cause the first elongate instrument 24 to advance or withdraw depending on the first translatory signal received from the input controller 12. The first elongate instrument 24 may be, for example, a guidewire.

The slave component 20 further comprises a second linear actuator 26, which is in electronic communication with the input controller 12 (see, e.g., FIG. 2C). The second linear actuator 26 receives a second translatory signal (not shown) sent from the input controller 12. The second linear actuator 26 is configured to cause a motion (translation) of a second elongate instrument 28. The second linear actuator 26 will cause the second elongate instrument 28 to advance or withdraw depending on the second translatory signal received from the input controller 12. The second elongate instrument 26 may be, for example, a catheter.

The slave component 20 further comprises a first rotational actuator 30, which is in electronic communication with the input controller 12 (see, e.g., FIG. 2A). The first rotational actuator 30 receives a first rotational signal (not shown) sent from the input controller 12. The first rotational actuator 30 is configured to cause a motion (rotation) of the first elongate instrument 24. The first rotational actuator 30 will cause the first elongate instrument 24 to rotate about a longitudinal axis depending on the first rotational signal received from the input controller 12.

The slave component 20 further comprises a force sensor 32 in electronic communication with the input controller 12. The force sensor 32 is configured to measure a force acting upon the first elongate member 24 on at least one degree of freedom (“d.o.f.”).

For example, if movement of the first elongate member 24 is attenuated by, for example, a constriction in the vasculature of the individual in which it is inserted, the force sensor 32 will measure the increased resistance to movement. The force sensor 32 will send a force signal (not shown) to the haptic feedback mechanism of the input controller 12. In this way, an operator of the input controller 12 will sense, through the haptics of the input controller 32, the increased resistance.

The slave component 20 may further comprise a mounting arm 34 (see, e.g., FIG. 2B). The first linear actuator 22, second linear actuator 26, and first rotational actuator 30 may be attached to the mounting arm 34. The slave component 20 may have a traveling cart 36 in slidingly attached to the mounting arm 34 such that the traveling cart may translate along a longitudinal axis of the mounting arm 34. A motor 38 may be affixed to the mounting arm 34 and in mechanical communication with the traveling cart 34 such that the motor 38 may cause the traveling cart to move relative to the mounting arm 34. The first linear actuator 22 and/or the first rotational actuator 30 may be attached to the traveling cart 36.

The first linear actuator 22 and/or the second linear actuator 24 may be a friction wheel device. As such, the actuators may further comprise two wheels 40, 42 to advance or withdraw the elongate instrument. The wheels 40, 42 may act against the instrument to force the instrument to move through the friction wheel mechanism. A motor 44 is in mechanical communication with at least one of the wheels 40 to cause rotation of the wheel 40.

The first rotational actuator 30 may further comprise a rotatable clamp 46. The rotation clamp 46 is configured to clamp and release the first elongate instrument 22 in order to rotate the first elongate instrument 22 along a longitudinal axis of the first elongate instrument 22. A motor 48 is in mechanical communication with the rotatable clamp 46 to cause the clamp 46 in order to cause the clamp 46 to rotate.

In another embodiment of a first rotational actuator, a wheel may be provided to act against the first elongate instrument and cause the instrument to rotate about its longitudinal axis. A motor is in mechanical communication with the wheel to cause the wheel to rotate.

The force sensor 32 may be an electrical sensor (not shown) coupled to the first linear actuator 22 to measure the load used to translate the first elongate instrument 22. In the case where the first linear actuator 22 is a friction wheel device, the electrical sensor may be in electrical communication with the motor of the friction wheel device in order to measure the power consumed by the motor.

In another embodiment of the force sensor 32, the force sensor 32 may be a six d.o.f. sensor in mechanical communication with the first elongate instrument 22. A six d.o.f. sensor may be configured to measure the forces acting upon the instrument 22.

A system 10 of the present invention may further comprise a fluoroscope 50 to provide radiographic images of the position of the first and/or second elongate instruments 22, 26. The system 10 may comprise a display 52 in electronic communication with the fluoroscope 50. The display 52 shows the images produced by the fluoroscope 50. In this way, a user of the system 10 is able to visualize the action at the end of the instruments 22, 26 in order to inform his operation of the input controller 12.

In another embodiment of a system 60 according to the present invention depicted in FIG. 35, the first linear actuator 62, second linear actuator 64, and first rotation actuator 66 may be affixed to a platform 68. The platform 68 may be affixed to an attachment end 70 of a robotic manipulator arm 72. A robot input controller 74 is in electronic communication with the robotic manipulator arm 72 and provides a positional signal to the arm 72. In this manner, a user operating the robot input controller 70 causes movement of the robot manipulator arm 72. This embodiment will enable the slave component 76 to be more easily positioned to access a port of the individual through which the instruments will be inserted.

The invention may be embodied as a method 200 (FIG. 36) for telerobotic endovascular intervention for inserting into the vasculature of an individual at least a first elongate instrument having a longitudinal axis and a second elongate instrument, the second instrument having a cavity through which the first elongate instrument may pass. The method 200 comprises the step of providing a system 203 similar to that described above. The second linear actuator of the system is used 206 to insert the second elongate instrument into the vasculature of the individual. The first linear actuator is used 209 to insert the first elongate instrument into the vasculature of the individual by way of the cavity of the second instrument. The first linear actuator is operated 212 to advance or withdraw the first instrument. The first rotational actuator is operated 215 to rotate the first instrument about the longitudinal axis. The second linear actuator is operated 218 to advance or withdraw the second instrument.

An input controller may be provided to allow a user to operate the actuators of the system. The method 200 may further comprise the steps of using 221 the input controller to cause the first translatory signal to be sent to the first linear actuator and moving 224 the first instrument according to the first translatory signal.

A system according to the present invention may be used with elongate instruments to deliver a medical device to a position within the vasculature of an individual. For example, an instrument may be used to deliver a stent within the individual. Such an instrument may have an end-effector at a distal end (the end which is inserted into the individual), and a mechanism to change the status of the end-effector (e.g., grasp or release) at the proximal end on the instrument. An end-effector actuator may be provided to operate the mechanism and thus operate the end-effector. A device may be provided at the console for actuating the end-effector actuator. The device may be, for example, a button on the input controller. The device may be in electronic communication with the end-effector actuator and send an operating signal to the end-effector actuator.

An exemplary system according to an embodiment of the present invention was built (called SETA). A description of the system follows. The description is not intended to be limiting, but rather to further describe an embodiment of the invention.

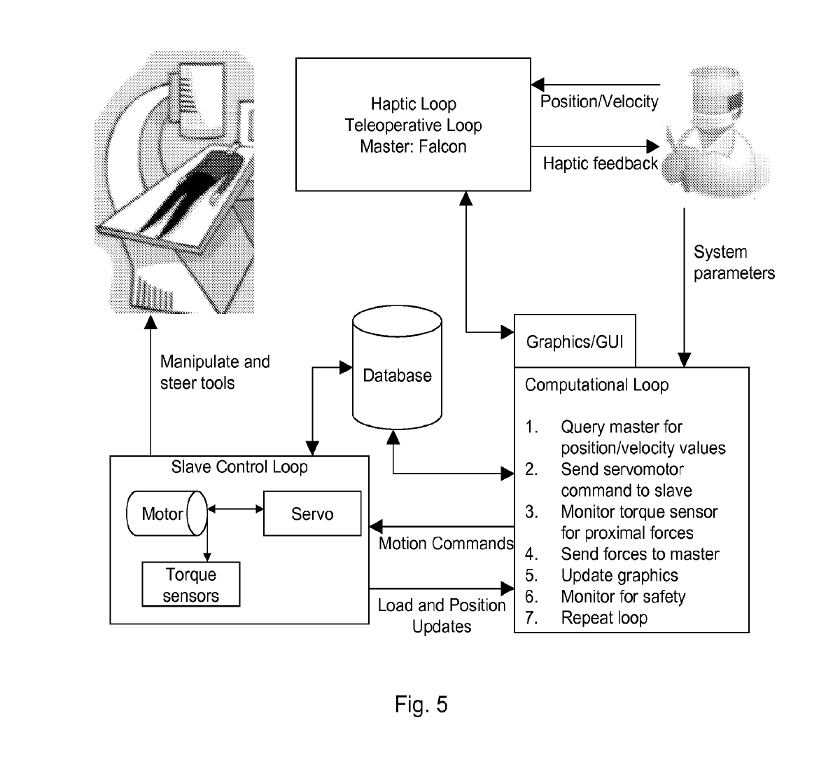

SETA comprises 4 components (FIG. 5), they are:

1. Patient side slave manipulator: This manipulator comprises of two translational and one steering stage; allowing for simultaneous manipulation of catheters and guidewires. The mechanism also has a force sensing framework used to actively monitor the safety of the procedure and provide force feedback to the surgeon.

2. Master controller: Novint's Falcon haptic device was used as the input mechanism to communicate position and velocity commands to the slave and at the same time provide force feedback to the operator.

3. Control module: The control module of the system includes the electronics used to drive the motors on the patient side slave and process sensor communication. This includes the computer that served as the mediator between master, slave and the user.

4. Algorithms: SETA has algorithms for haptic rendering, position and velocity control, teleoperation, motion scaling and tremor removal. These algorithms help interface the master with the slave and provide useful features for the operator.

Patient Side Slave Manipulator

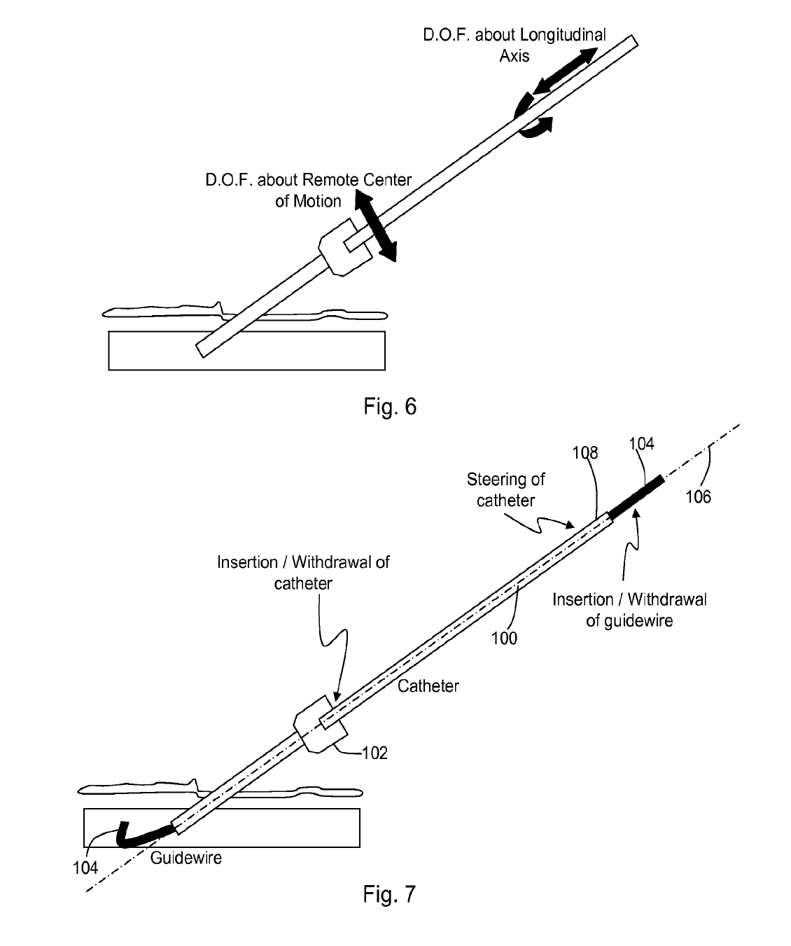

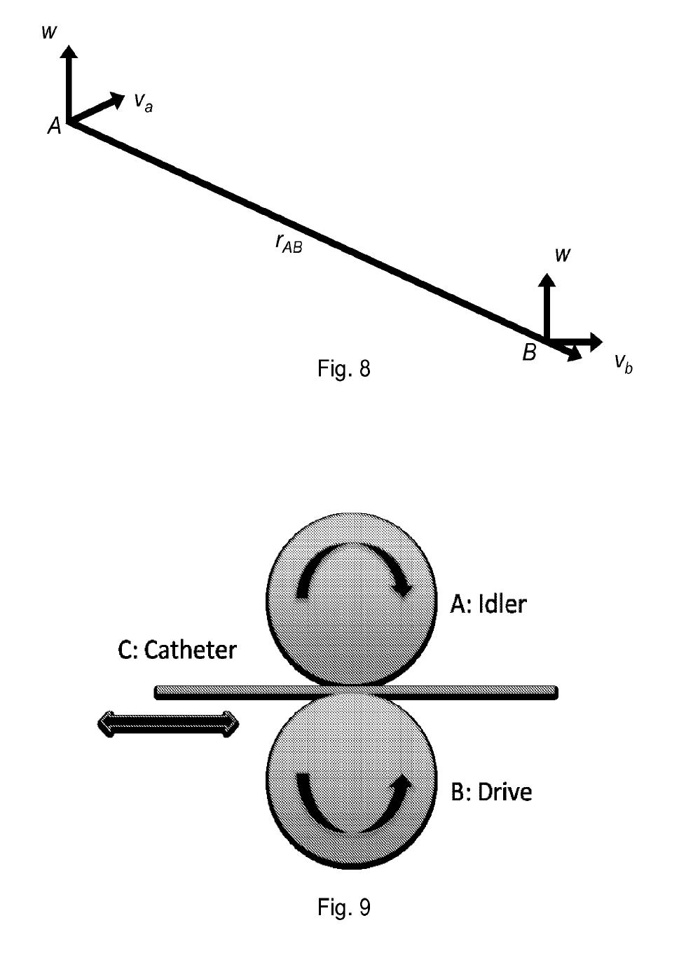

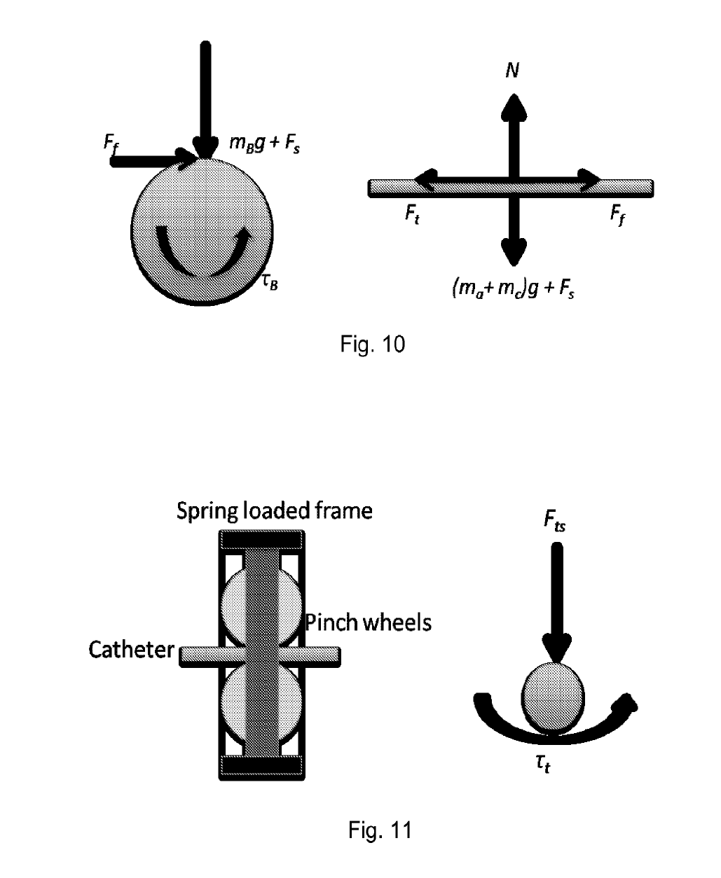

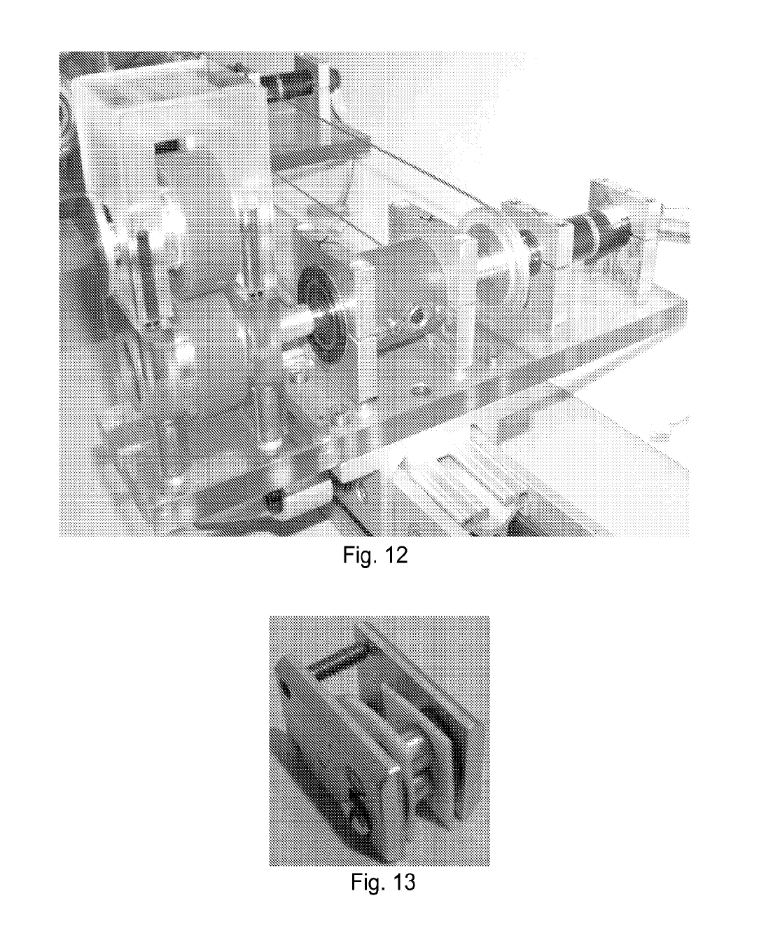

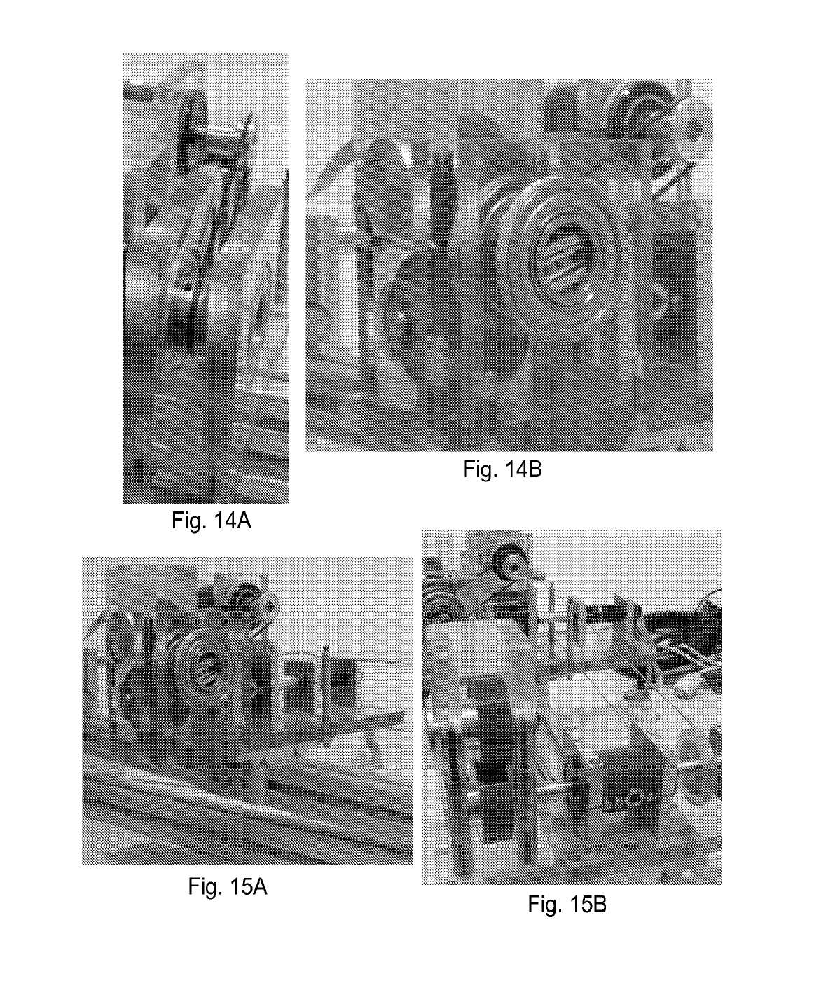

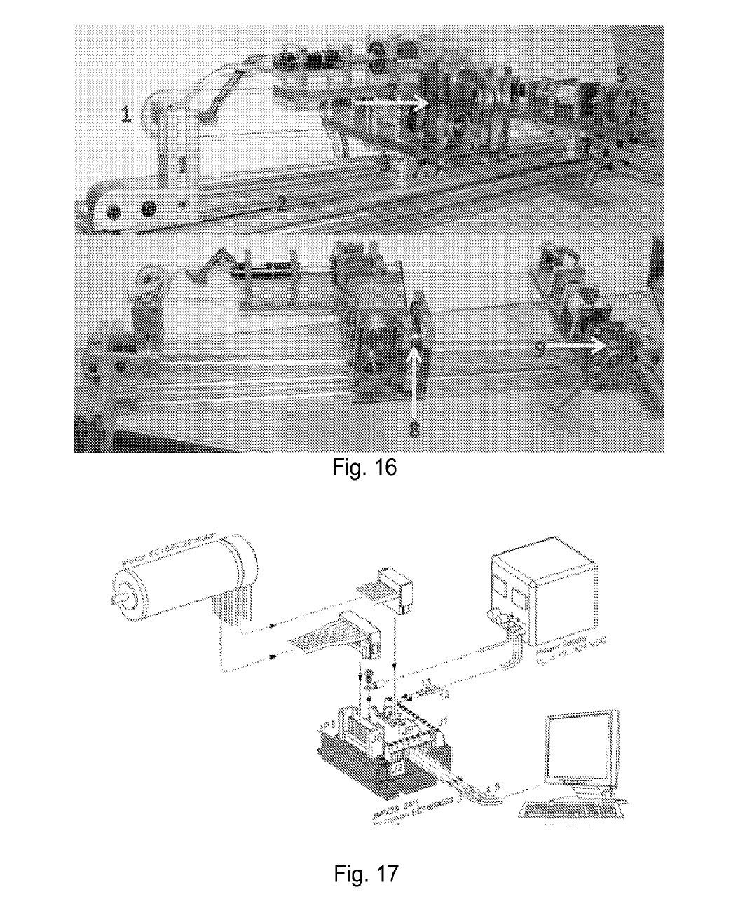

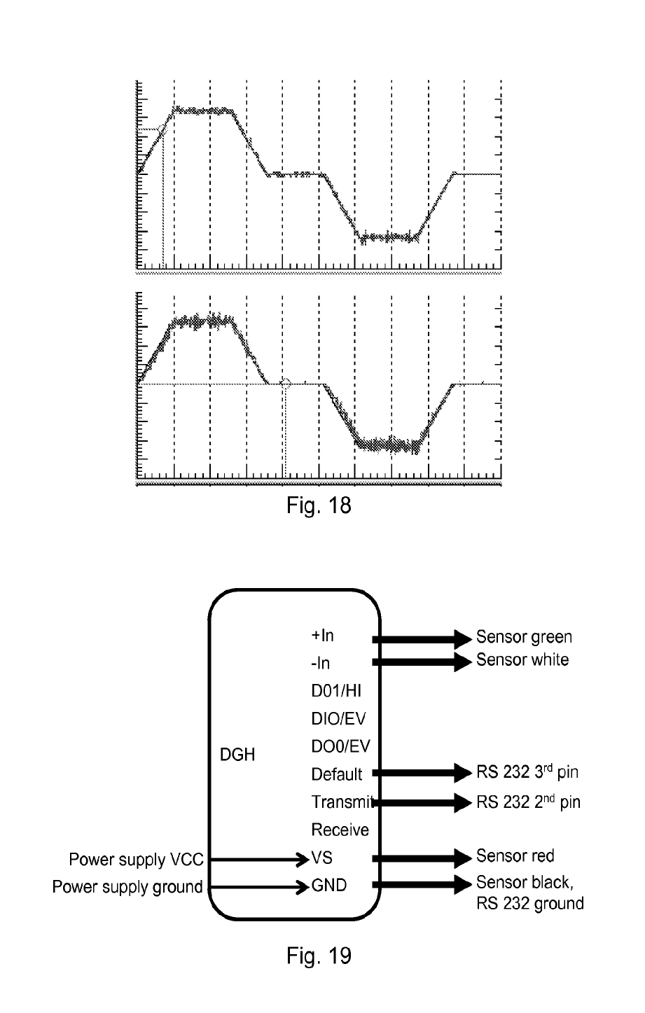

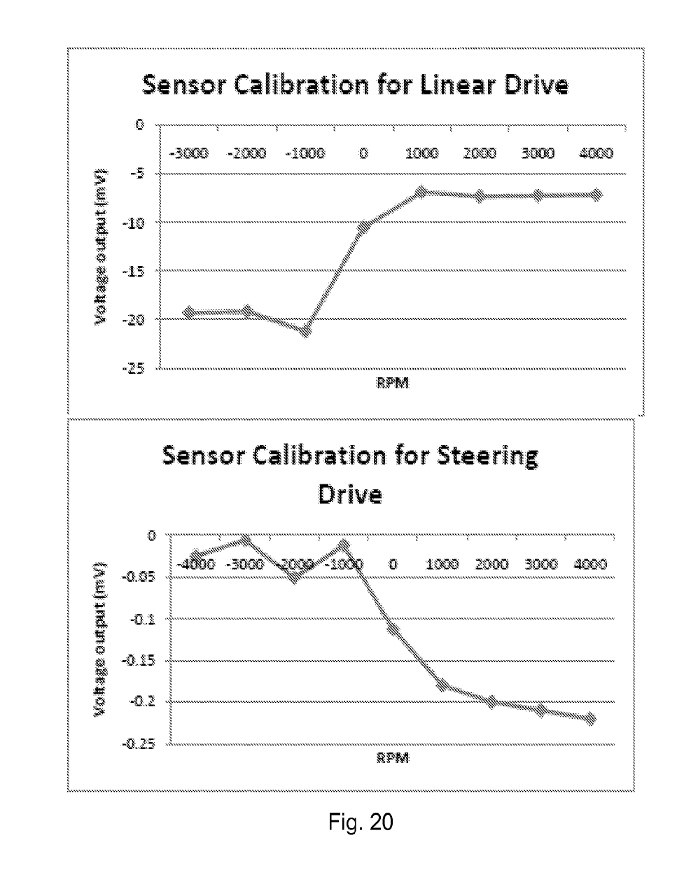

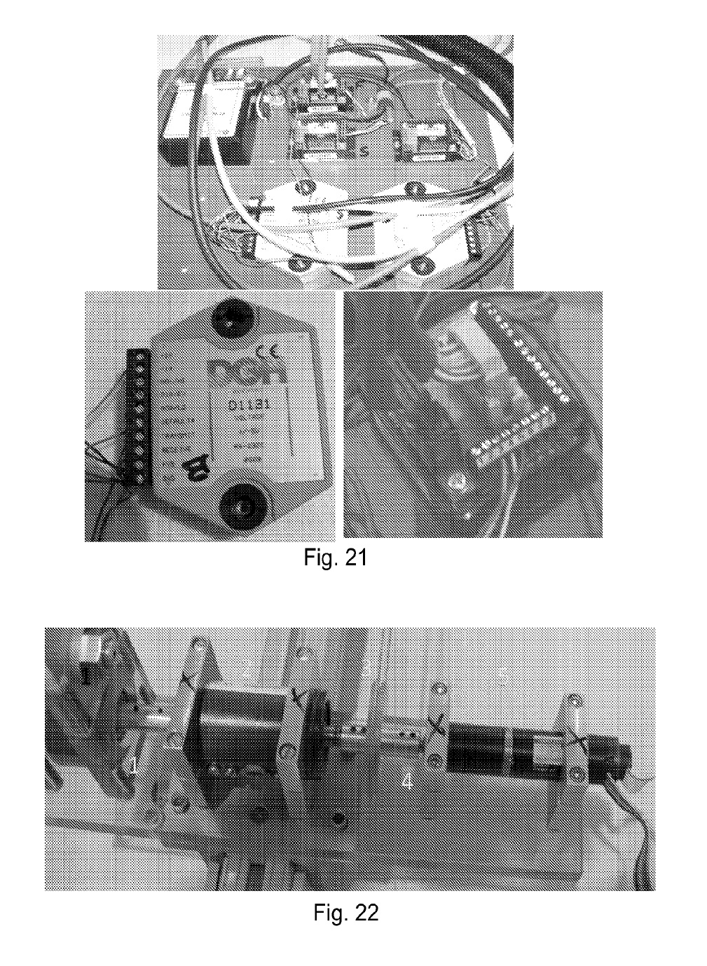

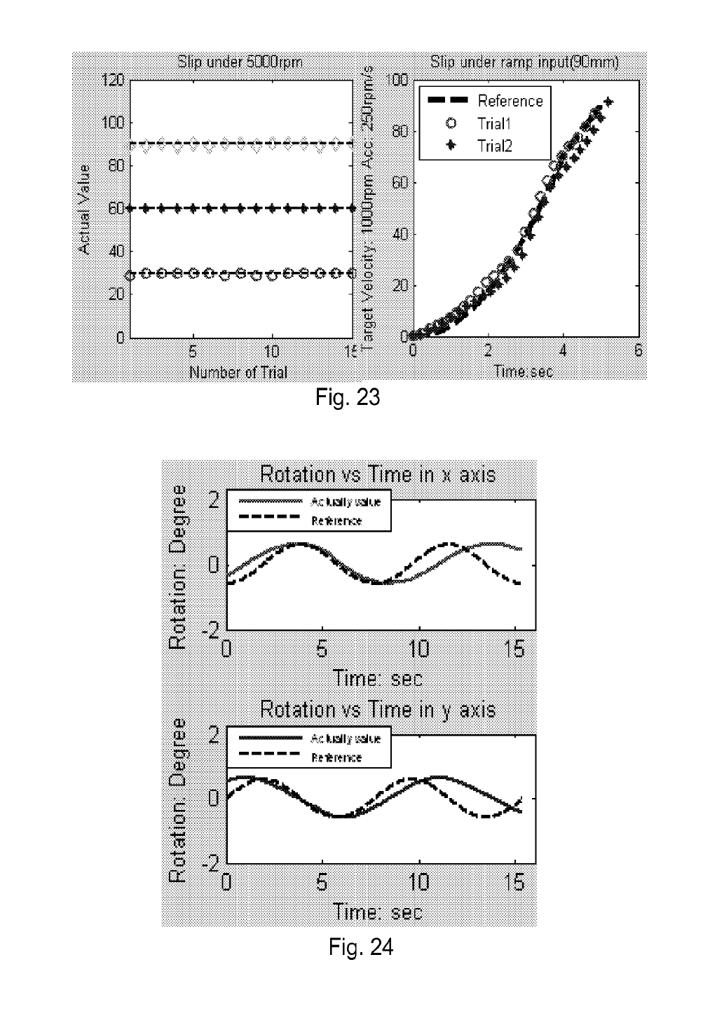

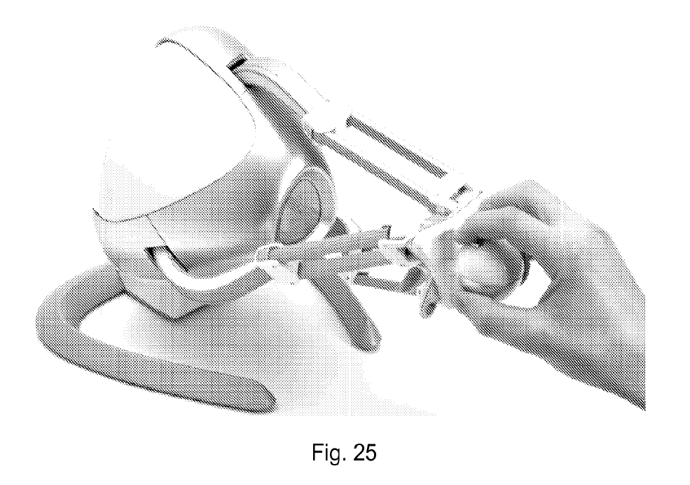

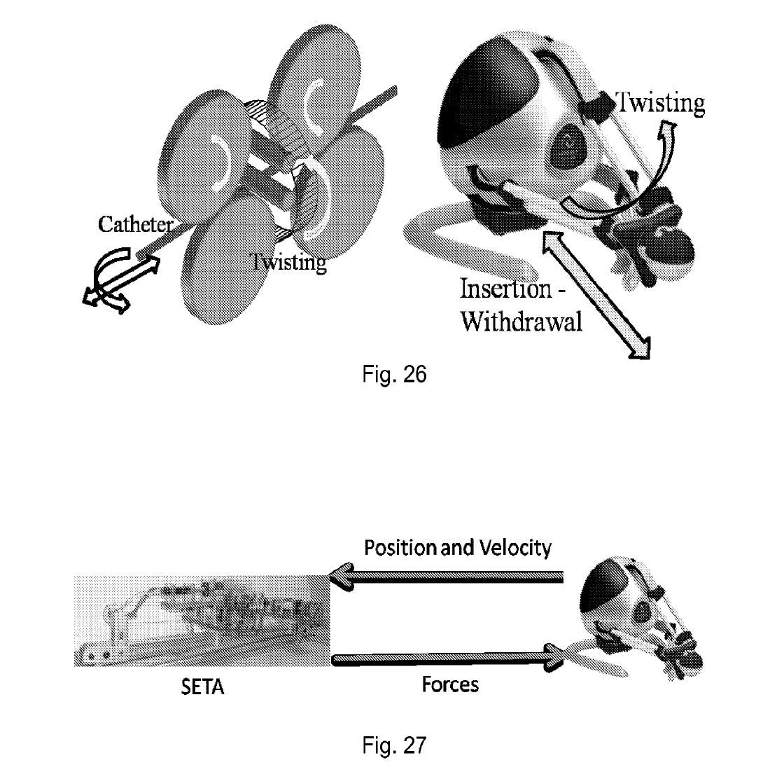

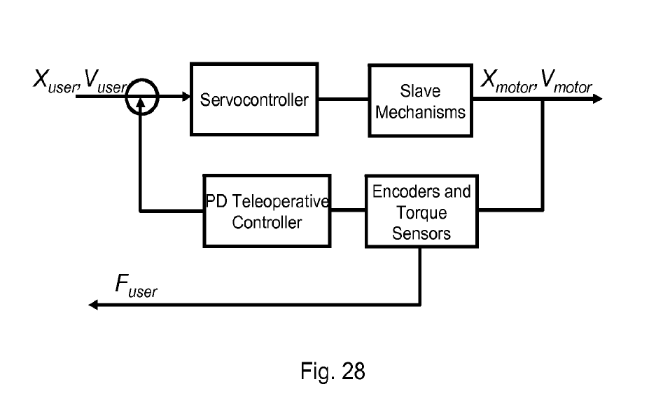

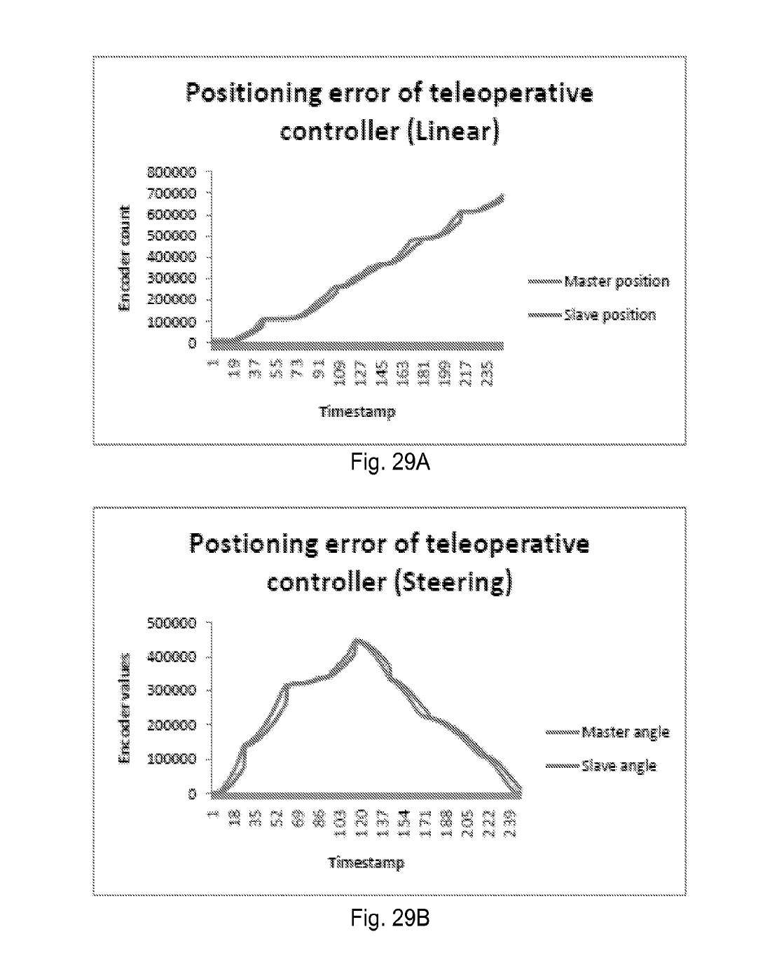

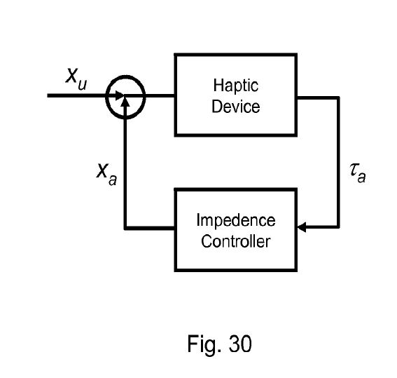

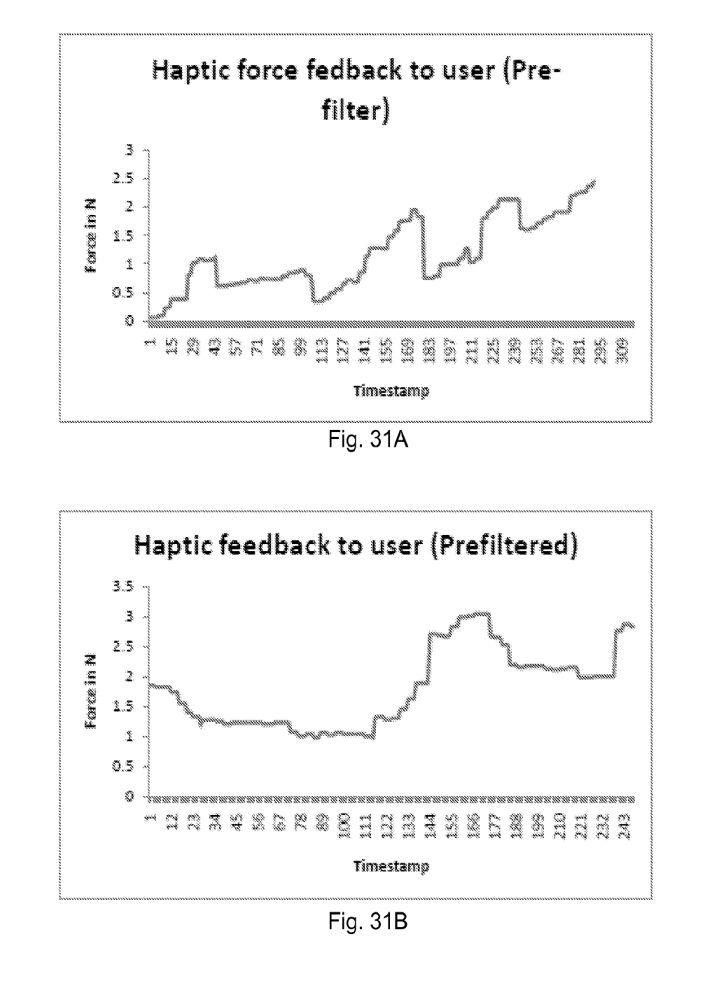

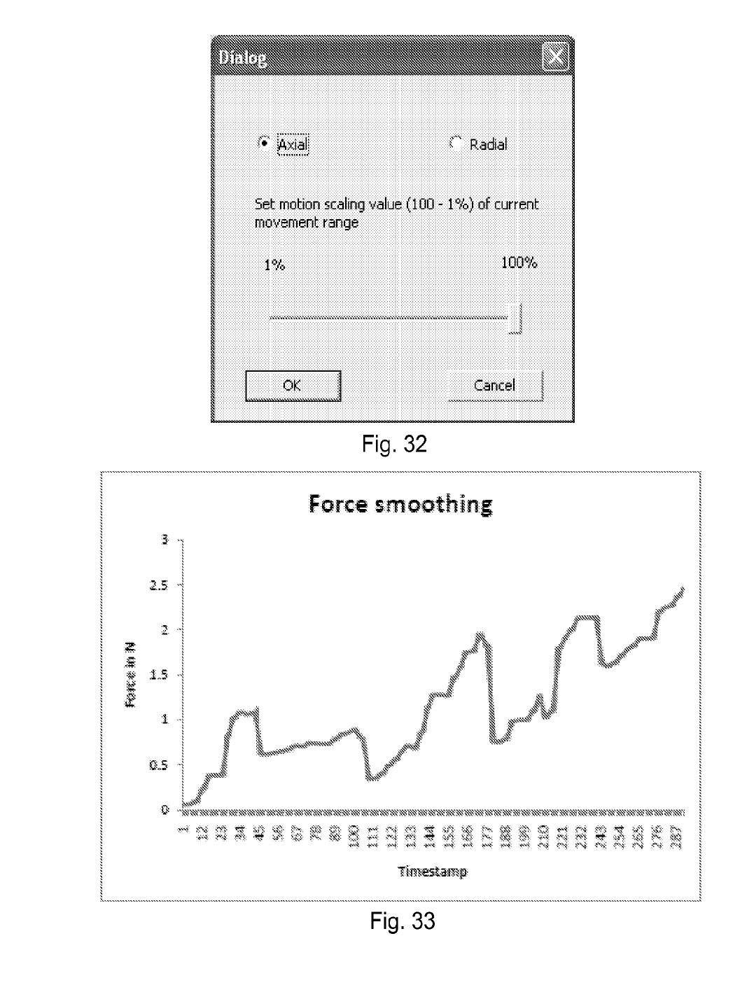

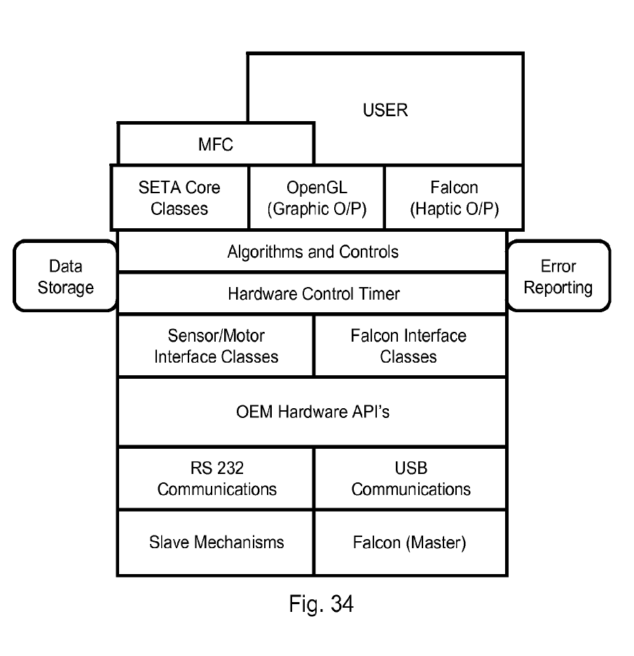

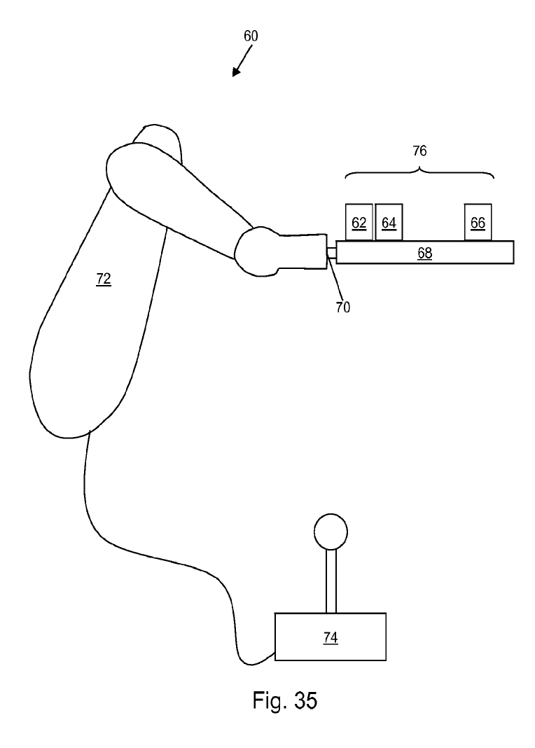

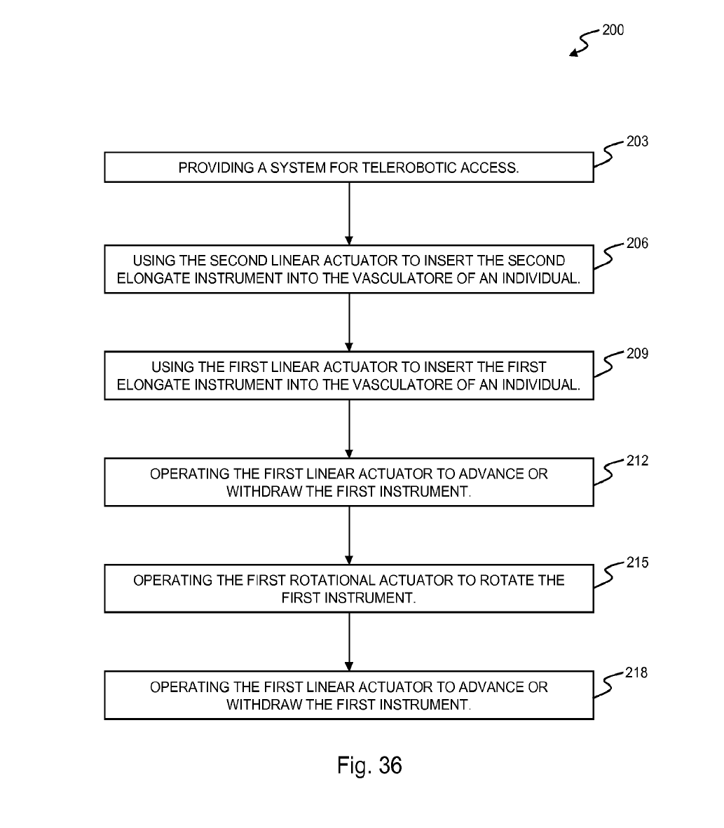

All Minimally-Invasive Surgery (“MIS”) procedures use ports to obtain access inside the body. As an effect of using ports, tools used for MIS procedures have at least two degrees of freedom on their longitudinal axis; a translation along that axis and a rotation about that axis (FIG. 6). Additionally, depending upon the procedure, the tip of the tool may have additional degrees of freedom through articulation (e.g., laparoscopy tools). For endovascular surgery the operator does not possess active control over the tool tip; instead, the operator uses the inherent dynamics of the tool and its interaction with the vascular geometry to guide it. The specific type and number of movements (insertion, withdrawal, and steering) needed for this form of surgery has been identified and experimentally confirmed to be distal actuation on the longitudinal axis, near the site of entry. Any slave mechanism designed for endovascular telesurgery must be capable of providing such distal actuation for two tools (a guidewire and a catheter) simultaneously.

Based on operating space, MIS procedures can be classified under two broad categories—procedures that are carried out inside body cavities using rigid tools (laparoscopy) and procedures carried out using lumen (endovascular interventions). In the former case, due to access through a fixed port and rigidity of tools, the tools have a pivot situated at the insertion point (port). This acts as a remote center of motion (“RCM”) and the tool has two additional degrees of freedom about this point (FIG. 6). As such, the tool behaves like a constrained spherical mechanism. In the latter case, due to the tool's (catheter, etc.) flexibility, these extra degrees of freedom are not observed. The design of the slave mechanism must have sufficient degrees of freedom to provide articulation matching the procedure as performed by a surgeon manipulating the tools directly. At a minimum, these additional degrees of freedom are required to line up the slave mechanism to an introducer sheath placed on the femoral artery. Due to high impedance and the flexibility of the tools at the point of entry, any misalignment can result in the tool buckling upon entry.

During the course of a procedure, the interventionalist manipulates the catheter 100 and guidewire 104 at three distinct points (FIG. 7). The catheter 100 is inserted and withdrawn near the cannula 102 providing translation movement along the catheter's 100 longitudinal axis 106. The interventionalist applies torque at the catheter hub 108, to steer the catheter 100. The catheter 100 is steadied near the cannula 102 with the non-dominant hand as the catheter 100 is being steered using the dominant hand. The guidewire 104 is provided translation movement at the point at which it enters the catheter hub 102. The catheter 100 is clamped at the hub 102 using the non dominant hand whenever the guidewire 104 is manipulated. With these requirements in mind, suitable articulation methods were designed for the slave manipulator.

Screw Theory

Screw Theory (“ST”) can be used to describe any displacement, which involves a translation and rotation of an elongated object about a single axis. Basic movement of all interventional devices falls under this theory and it can be used to model their motion. An issue that needs to be addressed is that interventional devices are highly flexible and ST is typically applied to just rigid bodies. However, a small section of an interventional device (FIG. 4) held in tension with no external forces acting between anchor points can be considered to quasi rigid. ST can then be used to model the kinematics of the device. Any finite translation Δx and rotation 0 about the tool's longitudinal axis can then be represented through the corresponding velocities using the twist equations (Equation 1). One of the strengths of the twist representations is that the two motion components can be decoupled into prismatic and revolute motions (Equation 2). In the screw equation, when the magnitude of either component tends to zero, the equations of motion separate out to that of a revolute and prismatic joint. As the guidewire is not steerable in the proposed slave, the kinematic equations for the guidewire are given by that of a prismatic joint. The decoupled kinematics of the system allows for use of simpler mechanisms that can be serialized easily to provide a complex resultant motion. To achieve manipulation at distributed points as shown in FIG. 3, an actuation chain can be constructed using three independent mechanisms at the identified points. In the equations there are: translational velocities at reference points (ν_{B}, ν_{A}), angular velocity of tool (ω), cross product between reference frames at points of interest (r_{AB}, twist in joint (t), point on link of interest for a revolute joint (q) and velocity of link in a prismatic joint (ν).

ν_{b}=ν_{A}−r_{AB}×ω

Equation 1: Description of Screw Motion

$$t = [ \begin{matrix}q \times ω \\ ω\end{matrix} ]$$

$$t = [ \begin{matrix}v \\ 0\end{matrix} ]$$

Equation 2: Decomposition of Screw Motion for Revolute and Prismatic Joints

Two types of drive mechanisms are required for the slave system; a linear mechanism, for providing translational motion, and a torquing or twisting mechanism, for providing steering motion.

Linear or Translational Drive Systems

The slave component comprises a friction wheel drive to provide translatory motion of a guidewire and a catheter. Friction wheels can provide an infinite stroke, have a relatively small construction, and do not suffer from ripple effects. The friction wheel mechanism may further be placed on a traveling cart which is moved linearly by a cable and pulley system.

Steering Drive Systems

The small and variable diameter of the catheter and guidewire consumable devices (0.014″-0.1″), their high flexibility, and varied material of construction, make the design of steering systems difficult. Friction wheels are mounted orthogonal to the longitudinal axis of the tool, may be used to provide torquing movement (rotation about the longitudinal axis).

In another embodiment of a slave component, a clamping system is used. A clamp may capture and twist a catheter or guidewire. These clamps may be biased by a spring to maintain gripping force on the catheter or guidewire. Clamps may feature rollers such that translational movement is not impeded. Clamps may be driven through through planetary gears and/or a pulley arrangement.

SETA

SETA's slave mechanism was designed as a two stage system (FIGS. 2A-2C), with a traveling cart that houses manipulators for actuating the guidewire (FIG. 2A) and steering the catheter (FIG. 2B) and a fixed mechanism (FIG. 2C) that was used to provide linear drive to the catheter. The linear actuation stages were constructed using friction wheels and the steering stage was constructed using a miniature gripper-pulley combination. The traveling cart was also actuated using a pulley arrangement. Both actuating mechanisms were driven using a brushless DC motor and a torque sensor was coupled between the load and the motor. The designed slave mechanism possessed three distinct manipulation points, corresponding to requirements provided in FIG. 7. The traveling cart ensures that the relative position of the catheter hub and guidewire are maintained throughout manipulation. The system can manipulate the devices in three separate modes.

1. Catheter: The catheter can be isolated for insertion and steering.

2. Guidewire: The guidewire can be isolated and provided linear drive.

3. Simultaneous: Both catheter and guidewire can be simultaneously provided translational motion, maintaining the relative tip positions. The catheter can be steered independently.

The system was mounted on a mounting frame that partially satisfied the requirements for RCM.

Motion Dynamics

Free body diagrams were constructed for the linear and torquing stages and dynamic equations were derived based on Newtonian principles. The equations were used with the values extracted from the design criterion to determine the power required from the motors, the dimensions and properties of the manipulators (friction wheels and gripper) and the transmission parameters for the pulleys.

For linear motion of tools, FIG. 9 gives the free body diagram. The three components under consideration are the idler and drive wheel, and the catheter being driven.

As described earlier, since the motion of the tool in two directions are decoupled, the dynamics can be modeled separately too. The free body force diagram for each component is given in FIG. 10.

$${I}_{B} {α}_{B} = {τ}_{B} - {F}_{f}$$

$${α}_{C} = {F}_{t} - 2 {F}_{f}$$

$$where , \\ {F}_{f} = ( {F}_{s} + {m}_{A} g ) μ$$

$${α}_{C} = \frac{{v}_{C}^{2} - {u}_{C}^{2}}{2 {x}_{C}}$$

$${α}_{B} = {α}_{C}$$

$${I}_{B} = \frac{{m}_{B} {r}_{B}^{2}}{2}$$

$${F}_{t} = \frac{{τ}_{B}}{{r}_{B}}$$

Equation 3: Derivation of Torque Requirement for the Linear Drive

From FIG. 10 (and Equation 3) it may be seen that the main forces acting on the catheter at this section were the tangential drive force (Fₜ), frictional force from interaction with the rollers (F_{f}) and the normal reaction at the manipulation point (N). The mass of the catheter can be assumed to be m_{c} and its linear displacement given by x. Similarly the forces acting on each of the wheels are the driving torque on B (τ_{B}), force from spring loading (F_{S}) for identical rollers properties (Inertia: I, mass: m, radius: r). From this, for given velocities of the tool (ν_{c}, u_{c}) torque requirements for the driving motor can be derived (Equation 3). Unknowns in the equation include the diameter of the friction wheel, spring force on the system and the friction coefficient of the interface.

A design using friction wheels was evaluated for providing torque or steering in the system (FIG. 3). Additionally, a new steering mechanism, consisting of a miniature gripper (FIG. 4) was designed. This mechanism behaved like an axial bearing that transmitted power radially, while allowing axial slip. This arrangement allowed smooth linear actuation of the tools and provided a smooth torquing action without any noticeable slip. The gripper in turn was driven using pulleys. Bearing parameters for maximum torque transmission with no slip and pulley parameters for power transmission from the motor required designing. As compared to the gripper assembly the mass of the catheter is negligible. Hence it is sufficient that the spring force (Fₜₛ) acting upon the catheter be balanced with the driving torque (τₜ) for a no slip condition. FIG. 11 shows a schematic of the system and its free body force diagram.

Equation 4: Minimum Condition for Slip or Lossless Torque Transmission from Motor to Interventional Tool.

Also, the spring force pinching the catheter for torquing mechanism (Fₜₛ) should be sufficiently larger than the spring force used for the linear drive (Fₛ). This condition would ensure transmission of torque through the linear drive. If this condition is not met, the linear drive would pinch the tool in place, not allowing transmission of torque. Similarly, consideration has to be given to the gripping force exerted by the gripper as the tool is given linear motion. This can be addressed through use of brass rollers with low coefficient of friction to allow smooth translation movement of the tools.

Linear Stage

FIG. 12 shows the current version of the linear drive. Based on the design equations and requirements, 2 inch diameter polyurethane friction wheels were used. The friction wheels were rated at Shore 55A hardness and provided a coefficient of friction approximately 0.6 with polyethylene. The same sets of wheels were used for both the roller and idler. The wheels have a keyway. The wheels were assembled on a custom made shaft using set screws. The drive was given to the lower wheel and the upper wheel acted as an idler. The wheels weighed 50 grams each and were calculated to have inertia of 0.017 kg/m2. The drive shafts were custom made using liz inch diameter steel barstock.

A custom housing was constructed to assemble and space the wheels. The housing was made using polycarbonate blocks and was constructed as two mating pieces. The lower assembly was constructed as two separate pieces to house the drive wheel. The pieces were bored and press fitted with suitable bearings (ball, ⅜ inch diameter) and assembled to the base plate supporting the system using ⅛th inch Allen head screws. The upper block was assembled as a single piece and had bearings to support a shaft on which the idler wheel was mounted. Holes were drilled on the top surface of the lower piece and they were press fitted with brass bushings. Similar mating holes were drilled into the upper assembly and steel roller pins were press fitted into them. The resulting assembly moves smoothly along the pin axis, creating a self adjusting system to accommodate interventional tools of different diameters without any external adjustments. Teflon spacers were created to reduce rubbing of the wheels with the housing. This friction wheel arrangement allowed use of tools with diameter from 0.014 inches all the way up to 10 Fr catheters (0.13 inches).

Choice of wheel diameter coupled with the maximum continuously variable speed of the servodrive allowed matching of recorded stroke lengths and velocities. Provisions were provided on the acrylic frame for mounting tension springs to provide a stronger gripping force on the tools. The weight of the entire assembly was calculated to be 130 grams, resulting in a normal load on the tool of 1.275 N. This was within range of pinch pressure applied by interventionalists during procedures and at the same time does not exceed the safety limitations set during the design requirements. Buckling of tool upon entry is a common problem encountered during interventions. Preferably, when the tool buckles it should happen outside the lumen, rather than inside. Buckling inside the lumen can cause undesired interaction of the tool with the vessels. With the linear drive system, when it is not in operation (inserting the tool), a backward force of 0.76 N is sufficient to cause the tool to slip in the reverse direction across the surface of the friction wheels. This value is approximately four times less than the maximum possible tool force of 3N. In this way, any buckling will happen outside the lumen and not inside.

Steering Stage

The steering stage was constructed in two parts; a miniature gripper (FIG. 13) and a pulley arrangement (FIGS. 14A-14B). The pulley arrangement drove a bushing based on the operator input. The miniature gripper was housed inside the bushing moving torsionally as the bushing moved. The gripper acted like a coupling, holding the tool in place as it was actuated through the bushing. The sequence of movements in net effect provided torsional movement to the tools. The pulley arrangement was connected to a drive train consisting of a servomotor and torque sensor, similar to what was used with the linear drives. The pulley was press fitted onto the sensor shaft. The drive pulley was 1 inch in diameter and was constructed using steel barstock and had a groove to take a 0.075 inch belt. The driven pulley was constructed using a brass bushing (1.25 inch OD 1 inch ID). A groove was machined into the bushing to run the belt. The bushing was mounted between two polycarbonate blocks that were secured on the traveling cart's base. The polycarbonate blocks were bored to house bearings for supporting the bushing. This allowed for a smooth and relatively frictionless movement. The miniature gripper was secured inside the bushing using a set screw. A hole was drilled and threaded on the bushing to house a nub set screw that held the gripper in place.

The miniature gripper was assembled using two aluminum frames. Steel shafts were pressed through the frame and brass roller pins were mounted on the shaft. One of the shafts rested in an elongated groove such that it could travel up to 0.11 inches. This travel allowed the gripper to accommodate tools of different dimensions. The shafts themselves were tension loaded using O rings. The tension provided by the O rings held the tool in place as it was being driven torsionally. Based on the material of the O ring and maximum elongation of the ring, it was calculated that a maximum of 3 N (Fₜₛ) of force would be applied on the tools. This force is larger than the load applied on the tool by linear stage (1.275 N) and hence ensures that the driven tool can overcome load applied by the friction wheels and propagate torque through its length. Teflon spacers were used to ensure that the rollers do not rub with the O rings or the aluminum housing. Set screws were used to maintain the structural rigidity of the gripper frame.

Traveling Cart and Positioning Pulley

A traveling cart was constructed for housing the catheter steering and guidewire insertion mechanisms. The traveling cart maintains the relative position of the manipulation points shown in FIG. 6 for these two mechanisms. The traveling cart was constructed by attaching the housing plate of the guidewire drive mechanism onto a linear slide. A pulley setup was constructed for actuating and positioning the cart. The driver pulley was located on the drive shaft of the catheter insertion mechanism. The pulley was constructed from a steel barstock and was 2 inches in diameter. The diameter of the pulley ensured that the traveling cart traveled the same distance as the length of catheter inserted/withdrawn. The driven pulley was fixed on the mounting arm on which the mechanisms were mounted. The driven pulley was constructed using high quality polycarbonate and 0.075 inch braided stainless steel cable was used for transmission of power. Two 2.5 inch Allen head screws were inserted onto the traveling cart and holes two inches apart were drilled into the screws. The pulley cable was passed through the holes in the bottom and was fixed into place using the set screws running through the hole on the top. Thus, as the pulleys are driven the traveling cart is pulled along the length of pulley cable. FIG. 15 shows the constructed traveling cart and the positioning pulley.

Mounting Arm

A mounting arm was constructed for housing the three mechanisms used to manipulate the interventional tools. Apart from providing structural support and housing for the mechanisms, the mounting also served as a passive method of providing compliance with remote center of motion requirements. Through adjustment of the mounting arm's links, desired elevation angles can be reached at the point of insertion into the lumen. The mounting arm itself can be positioned to achieve the necessary azimuth. The mounting arm has an initial incline of 15 degrees, which was considered a suitable angle for insertion of tools into the lumen. Other angles of inclination may be used to prevent buckling of the tools on insertion into the lumen.

The mounting arm was constructed using 1.5 inch square aluminum extrusions. The manipulator was fixed permanently at one end of the mounting arm with the traveling cart free to move along the incline. FIG. 16 shows the complete assembly of the patient side slave manipulator, along with the mounting arm.

Motor

EC max 22 brushless DC motors from Maxon Motors Inc. were used as actuators for mechanisms on the slave. These motors are rated for a maximum continuous torque of 22.9 Nm, with a maximum permissible rating of 18000 rpm. The motors work at 24 VDC with a peak current of 1.41 A. An optical encoder (Encoder MR, type M) was mounted onto the motor shaft. The encoder has 512 counts per term, 4 quadrants of operation (cumulative of 2048 per turn) and 3 channel communication. A gearhead was used to step up the motor torque and step the down it's rpm. The gearhead (GP 22C) has a 1:128 reduction ratio, providing approximately 3 Nm of continuous torque output on the shaft.

FIG. 17 shows the cabling used for connecting the controllers, and the wiring diagram for the system. The motor was connected to the EPOS Freedom 2411 series servocontroller (302267). Each servocontroller is capable of controlling one motor at a time and has inputs to the hall sensor and encoder on the motor. The servocontroller requires a power supply of 24 VDC with a maximum current of 2 A. It also supports up to six independent digital and analog inputs and outputs. This can be used to support motor auxiliaries. The servo features various control modes, including velocity control, position control and profile based position, velocity controls. The profile based velocity control uses a trapezoidal profile using user set acceleration and deceleration values to achieve positioning commands. Specific velocity values can be set for the profile position mode, thus this modality was used to set the velocity and positioning behavior of the slave mechanisms. Each motor was calibrated using regulation tuning to determine the optimal values for the PID gains for the servocontroller. Calibration and tuning graph can be seen in FIG. 18. The Freedom 2411 uses RS-232 communication for programming the controllers and executing commands. The standard cabling provided by the company is not directly usable for setting up the RS 232 communication or hooking up the encoder to the controller. To connect the encoder to the controller, custom cabling components were purchased and connected to a standard T-10 Ethernet cable to the system. Similarly the serial port connector had to be removed and internal cabling was used directly to connect the controller to the PC.

Maxon motors provides a C++ dll library (EposCmd.dll) for authoring custom applications to interface and control the servocontrollers-motors. The library provides functions to select, open and initialize a serial port at a given baud rate to communicate with the controller. This was followed by setting up a communication protocol for working with the device. Depending on the chosen control mode, there are a number of separate functions that allow setting of various command parameters for actuation. The library provides easy access to any fault states encountered by the system and it is communicated through a code or through the LED indicators present on the controller.

Proximal Force Sensing

LXT 971 torque sensors from Cooper Instruments Inc. were coupled to each of the servo drives and used to monitor the load on the drive units. The torque sensors are rated +/−2.5 N-m with a resolution of 0.05 N-mm. The sensor comes with a signal conditioner and controller (DGH 1131). The DGH unit can be used to stream the sensor data through a RS 232 port to a PC. The unit has an EEPROM internal memory that allows for rudimentary programming and extraction of conditioned sensor data. The DGH is connected to the sensor through a special cable that had to be modified to connect to individual DGH ports. A separate RS 232 cable was purchased, the connectors removed and manually wired to the DGH. FIG. 19 shows the wiring diagram for the DGH-Sensor-PC setup. The system requires an external power supply unit to provide 10 VDC and 400 mA of current for every DGH-sensor combination. A single power adapter provided by Cooper instruments was used to drive two sets of sensor units.

Cooper Instruments Inc. provides a separate dll, (DGH_comm.dll) to collect data from the application. Using this dll, incoming data, in the form of voltage values (millivolts), were collected and used to calculate the load on the wheels. This information was used to derive the proximal load experienced on the catheter and other interventional devices. The calibration of the device was carried out in-house, using the motor assembly and a braking arrangement. The results of the calibration can be seen in the graph in FIG. 20. The sensor was calibrated under no load condition for varying RPM values. It was seen that both sensors suffered from a dead zone (˜−7.5 mV for linear drive and −0.075 mV for the steering drive). Beyond the dead zone, the sensor showed little variation in load for different rpm values. The dead zone may be attributed to the friction and inertial load of the system. Equation 5 was used to provide the final haptic feedback, where γ_{A} is the diameter of the driving wheel, DZ is the dead zone factor and ZA is the zero adjustment for the sensor. 0.737 represents the factor required to convert the sensor output into N.

$${F}_{feedback} = \frac{0.737}{{r}_{A}} = {sensor}_{reading} - ( DZ + ZA )$$

Equation 5: Computation of haptic feedback forces based on load reported by sensors.

Electronics and Other Components

FIG. 21 shows the electronics that were used to control and communicate with the motor and the sensor. The control accessories and their power supplies were mounted onto a 0.2 inch steel plate and secured in place using screws. The electronic components were grounded using the steel plate. The servo-controller had two inputs (one for the hall sensor and the other for the motor) that were entwined using tie wraps. The servocontroller had one output to the computer (RS 232 cable) and two leads providing VCC and ground. All RS 232 cables were secured using tie wraps and were enclosed in a flexible cable shield. The DGH sensor modules had one input coming in from the sensor and one output (RS 232) going to the PC.

FIG. 22 shows the drive train transmitting power from the motor to the load unit (friction wheel/pulleys). The sensor-motor shafts and the sensor-load shafts were secured using a sleeve that had key way. The sleeve also had set screws for maintaining positive drive and allow for easy assembly of the system.

Calibration and Testing

The performance of actuator chain can be detailed through multiple criteria. Some evaluation criteria examined for the slave system were:

Stroke: The stroke of the actuator chain, represents the total displacement range through which it can linearly actuate a device. For the actuation of guidewire and catheter, the use of friction wheel provides it with an infinite stroke length for both insertion and withdrawal. Similarly, there is no limitation of stroke or twist limits on the steering drive. However, the traveling cart arrangement restricts the working stroke or the length of catheter that can be actively manipulated by the user to 25 inches. This stroke length was verified by running a simple positioning experiment and taking measurements using a measuring tape. To ensure smooth working of the system, the traveling cart has to be manually reset to home position and calibrated before commencement of operations. The traveling cart can be initialized anywhere along the length of the mounting arm. This arrangement brings the convenience of an increase in stroke length with a longer mounting arm. The swivels on the mounting arm can accommodate stems providing stroke up to 35 inches in length.

Accuracy and Precision: The servodrives uses encoders with a resolution of 2048 counts/revolution. This gives an accuracy of (PI*Diameter of driving wheel)/2048 for the linear drive. This gives a translational positioning resolution of 0.003 inches. For the steering drive, as the tool is coaxial to the driven pulley the positioning accuracy of the system is given by (2*PI)/2048, which 0.003 radians. However, the actual system also has to account for losses due to friction and slip. To test this, the linear drive was given a series of step inputs to check for position accuracy and a ramp input to test for velocity. The linear drive was loaded with a 0.035 inch guidewire and 5Fr Boston Scientific Expo catheter and was given step input commands to move to 30, 60 and 90 mm at 1000, 2500 and 5000 rpm. Each combination of insertion length and speed was repeated 15 times and actual length of tool moved was measured. In the second experiment, the device was moved under ramp inputs to achieve insertion lengths of 30, 60 and 90 mm while accelerating from 1000 to 5000 rpm. It was found that for cumulative trials the mean slip or error in insertion was less than 0.4 mm for 100 mm length of insertion. The results of these experiments can be seen in FIG. 23. To test the steering mechanism, the aurora magnetic sensor was mounted on the bent tip of a 5F catheter. The tip of the catheter was then moved in 1 degree increments for 360 degrees at speeds of 1000, 2500 and 5000 rpm for 10 cycles. The experiment was repeated in both clockwise and counterclockwise directions and results can be seen in FIG. 24. A lag can be seen in the readings taken for the steering mechanism. The lag can be a result of two reasons. First, most interventional tools do not transmit torque in a 1:1 ratio from tip to tip. A certain amount of torque is stored as internal energy by the tool. Second, the measurement of the tip was performed indirectly, that is, using a magnetic sensor mounted on the bent tip of the manipulated tool. There is always a chance that the sensor and the tool tip were not moving in synch.

Safety: The patient side slave manipulator has a number of features that ensure the safety of the operator and the patient. They are:

1. Velocity: The system monitors the velocities of manipulation and if they exceed preset limits, the system will disconnect and provide an error message to the operator asking them to slow down.

2. Forces: The system monitors manipulation forces and cuts off the slave from the master whenever the manipulation forces exceed 3N in the axial direction and 6 N·mm in the radial direction. An error message is popped up to the user indicating that safe force limits were exceeded.

3. Manipulation: To avoid collisions with the mounting frame, a one inch buffer is provided for the movement of the traveling cart. Once the buffer zone is reached the system will disable the slave mechanism and will not allow further manipulation of the catheter in that direction until the cart is reset to home position.

4. Emergency cutoff: The system features a menu button for quick and easy access, which will perform an emergency switch-off of all actuators on the system. The actuators will be automatically turned off in the event of any adverse error on the servo drive too.

5. Force feedback: The master controller features an algorithm that has a saturation limit and a filter to remove any sudden or upward increases in forces feedback to the operator.

Master

Novint's Falcon haptic device (FIG. 25) was chosen as the master for this application. Falcon is a low cost haptic device that is robust and easily programmable. The Falcon is capable of delivering forces (0-8N) which is more than the range of forces (0-3N) required to simulate the feel of carrying out the procedure. The Falcon has a work volume of (4×4×4 inches) which is greater than the experimentally recorded maximum hand motion used by a surgeon. The system has a position resolution of 400 dpi, which translates to position resolution of 0.0025 inches. This is within the range of the positioning resolution of the slave manipulators. The haptic device connects to a PC through a standard USB port and is internally emulated as a serial device.

Mapping with Falcon

The Falcon features 3 d.o.f Cartesian movement of its stylus/handle. It does not provide twisting or rotational d.o.f. Thus, the natural hand motion of the surgeon (and the resultant tool movement), which involves steering and translation about the longitudinal axis, has to be mapped to the d.o.f available in Falcon. This was mapped as shown in FIG. 26. In-out movement of the handle along Falcon's Z axis was mapped to insertion and withdrawal movement of the tools. Left-right movement of the handle (along Falcon's X axis) was mapped to counterclockwise steering of the tool and Right-left movement of the handle was mapped to clockwise steering of the tool. As a safety (and operator error reduction feature) the four buttons available on Falcon's handle were used as switches to enable movement. That is, the correct switch had to be pressed and the handle moved in that direction for the slave to actuate the tool in the mapped direction (Table 1).

TABLE 1
Operator controls and resultant action on slave.

| Slave motion | Equivalent master command |
|---|---|
| Catheter/guidewire insertion | Press forward button on handle and |
| | move handle along negative Z axis. |
| Catheter/guidewire withdrawal | Press back button on handle and move |
| | handle along positive Z axis. |
| Counterclockwise steering | Press right button on handle and move |
| | handle along positive X axis. |
| Clockwise steering | Press right button on handle and move |
| | handle along negative X axis. |

Teleoperation

FIG. 27 shows the teleoperative schematic between the slave manipulators and the haptic master. As the operator pressed the correct buttons and moved the master handle, handle/hand position and velocity values were recorded at 500 HZ, filtered and forwarded to the slave as positioning commands/control input. The slave mechanisms carry out the commands and record proximal loads encountered during manipulation. These values are then provided as feedback to the operator through the haptic device. The teleoperation is unilateral, that is there is no update of the haptic device's position or velocity based on the location of the tool. During manual manipulation of tools, the interaction with the tool is in the form of discreet strokes and there is not continuous transmission of force values. As the tool's inertia and mass are magnitudes lesser than the hand actuating it, the forces provided by the tool are purely perceptual in nature. They are not sufficient to reposition the hand actuating it. Additionally, the tool has a tendency to buckle much before such a stage is reached. Unilateral teleoperation was chosen as the mode of operation based on these factors. For unilateral teleoperative control, it is sufficient to have a simple PD controller that will promise convergence of the slave's state vectors with that of the master (position and velocity). For this it is important the slave robot be compensated for gravity, Coriolis forces, and friction. By design and construction, the effect of gravity and Coriolis forces on SETA is minimal. The only dynamic effect that needs compensation for are friction and some inertial artifacts. These can be filtered out and compensated for by monitoring the sensor feedback and applying. FIG. 28 shows the implementation of the teleoperative system.

To illustrate the level of control achieved during teleoperation, a simple positioning experiment was conducted. A 0.035 inch guidewire was inserted and steered inside a vascular phantom. The results of teleoperation with respect the reference encoder position and true values for insertion and steering are provided in FIG. 29.

Even though the system is teleoperated, the master and slave are resident on the same system and share computational resources. Thus with good hardware and proper communication protocols the time delay in operation can be reduced to a value very close to zero. Thus there is no need to compensate for time delays in operation.

Haptic Feedback

Novint does not provide support for haptic rendering. The API supplied provides support for setting the force values directly. Hence an impedance control scheme was developed and integrated for the Falcon. FIG. 30 gives the schematic for the control scheme. Impedance control was chosen because haptic device does provide the actual force applied on the end effector. Thus, the control scheme has to be designed such that the robot adapts its compliance to objects encountered in its task space. Thus, any error in position of the robot's task space can be converted into an equivalent mechanical force exerted on (or by) the object due to change in compliance (Equation 5). In equation 5 we have the torque supplied by the actuator for a positioning command (τ_{actuator}), the torque developed in joints due to operators interaction with environment (τᵤₛₑᵣ), a second order representation of a mechanical system (m\ddot{x}+e\dot{x}%+k) and a stiffness based controller to correct error in the end effectors position (−K_{impedance}),

τ_{actuator}+τᵤₛₑᵣ=m\ddot{x}+e\dot{x}+k

τ_{actuator}=−K_{impedance}x

Equation 5: Impedance Control Equations.

The impedance controller was used to construct virtual planes to overall user movement to a bounded prismatic volume within the haptic device's workspace. As a result, the majority of an operators hand movements are within the X-Z plane with minimal movement in the Y direction.

The force feedback to the operator during procedures was based on the load experienced by the slave during manipulation, captured using the torque sensor (in millivolts) and converted to axial and radial forces (Newtons). The axial forces were fed back as the resistance experienced when the surgeon inserted or withdrew the tool (linear motion). The radial forces represented the resistance forces experienced when attempting to provide twist motion to the tool (through left-right movement of the master). FIG. 31A-31B shows the typical haptic forces experienced by a user as they try to advance a guidewire through a vascular phantom. It may be seen that the force fed back increased with insertion length, which can be attributed to increase in frictional forces and due to increased stiffness of the wire. Each distinct peak (FIG. 31A) represents an insertion movement of the tool by the operator.

Motion Scaling and Tremor Removal

To provide convenience of operation, motion scaling, tremor removal and force smoothing was added as part of teleoperation.

Motion scaling: A provision was added for scaling of all user movements down to 1% of actual movement value recorded by the master. The scaling was linear and made available for both the linear and steering stages. A dialog menu was used to set the scaling values for the master, where the actual values could be adjusted through a slider bar input (FIG. 32).

Tremor removal: Hand tremor and high frequency artifacts were removed from the position and velocity vectors recorded from the master through the use of low pass filters. A weighted moving average filter with a window width of five time-steps was used for data conditioning.

Force smoothing: Forces supplied to the operator were filtered using the weighted moving average filter to avoid sudden variation in haptic feedback. This would help ensure operator safety and providing the operator with a smooth haptic experience. FIG. 33 shows the results of force smoothing.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A system for endovascular telerobotic access for manipulating a first elongate instrument and a second elongate instrument, the elongate instruments having a common longitudinal axis, comprising:
a console comprising:
an input controller having a haptic feedback mechanism able to provide force feedback to an operator, the input controller providing a first translatory signal, a second translatory signal, a first rotational signal, and a second rotational signal; and
a slave component comprising:
a first linear actuator in electronic communication with the input controller for translatory motion of the first instrument according to the first translatory signal;
a second linear actuator in electronic communication with the input controller for translatory motion of the second instrument according to the second translatory signal;
a first rotational actuator in electronic communication with the input controller for rotating the first instrument about the longitudinal axis according to the first rotational signal; and
a force sensor in electronic communication with the input controller, the force sensor measuring a force in at least a first degree of freedom, wherein the force sensor provides a force signal to the haptic feedback mechanism of the input controller.

2. The system of claim 1, wherein the slave component further comprises a mounting arm, and wherein first and second linear actuators and the first rotational actuator are attached to the mounting arm.

3. The system of claim 2, wherein the slave component further comprises:
a traveling cart slidably attached to the mounting arm;
a motor affixed to the mounting arm and in mechanical communication with the traveling cart for translating the traveling cart along a longitudinal axis of the mounting arm; and
wherein the first linear actuator and the first rotational actuator are attached to the traveling cart.

4. The system of claim 1, wherein the first and second linear actuators each further comprise:
a friction wheel device having two wheels which rotate against the respective instrument in a forward direction to advance the instrument, and in a reverse direction to withdraw the instrument; and
a motor in mechanical communication with the friction wheel device to cause at least one of the two wheels to rotate.

5. The system of claim 1, wherein the first rotational actuator further comprises:
a rotatable clamp which grasps and releases the first instrument to rotate the first instrument about the longitudinal axis; and
a motor in mechanical communication with the rotatable clamp to cause the clamp to rotate.

6. The system of claim 1, wherein the first rotational actuator further comprises:
a wheel which rolls against the first instrument to rotate the first instrument about the longitudinal axis; and
a motor in mechanical communication with the wheel to cause the wheel to rotate.

7. The system of claim 1, wherein the force sensor is an electrical sensor coupled to the first linear actuator to measure the load used to translate the first instrument.

8. The system of claim 1, wherein the force sensor is a six degree of freedom sensor in mechanical communication with the first instrument to measure the forces on the first instrument.

9. The system of claim 1, further comprising:
a fluoroscope providing images of the first or second instrument;
a display in communication with the fluoroscope to display the provided images.

10. A method for telerobotic endovascular intervention for inserting into the vasculature of an individual at least a first elongate instrument having a longitudinal axis and a second elongate instrument, the second instrument having a cavity through which the first elongate instrument may pass, the method comprising the steps of:
providing a system comprising:
a first linear actuator for translatory motion of the first instrument according to the first translatory signal;
a second linear actuator for translatory motion of the second instrument according to the second translatory signal; and
a first rotational for rotating the first instrument about the longitudinal axis according to the first rotational signal;
using the second linear actuator to insert the second elongate instrument into the vasculature of the individual;
using the first linear actuator to insert the first elongate instrument into the vasculature of the individual by way of the cavity of the second instrument;
operating the first linear actuator to advance or withdraw the first instrument;
operating the first rotational actuator to rotate the first instrument about the longitudinal axis; and
operating the second linear actuator to advance or withdraw the second instrument.

11. The method of claim 10, wherein the step of providing a system further comprises an input controller operable by an operator, the input controller in electronic communication with the first and second linear actuator and the first rotation actuator, and the input controller providing a first translatory signal, a second translatory signal, a first rotational signal, and a second rotational signal.

12. The method of claim 10, wherein the step of operating the first linear actuator further comprises the steps of:
using the input controller to cause the first translatory signal to be sent to the first linear actuator; and
moving the first instrument according to the first translatory signal.

13. A system for endovascular telerobotic access for manipulating a first elongate instrument and a second elongate instrument, the elongate instruments having a common longitudinal axis, comprising:
a console comprising:
a instrument input controller providing a first translatory signal, a second translatory signal, a first rotational signal, and a second rotational signal; and
a robot input controller providing a positional signal; and
a slave component comprising:
a robotic manipulator arm having an attachment end, the robotic manipulator arm in electronic communication with the robot input controller;
a platform affixed to the attachment end of the robotic manipulator arm;
a first linear actuator affixed to the platform and in electronic communication with the input controller for translatory motion of the first instrument according to the first translatory signal;
a second linear actuator affixed to the platform and in electronic communication with the input controller for translatory motion of the second instrument according to the second translatory signal; and
a first rotational actuator affixed to the platform and in electronic communication with the input controller for rotating the first instrument about the longitudinal axis according to the first rotational signal.
